(19) United States
(12) Patent Application Publication
(10) Pub. No.: US 2006/0234977 A1
(43) Pub. Date: Oct. 19, 2006

(54) Antiviral compostion comprising a sulphated glucose polymer and a bacteriostatic agent

(76) Inventor: Gina Wenham, Liverpool (GB)

Correspondence Address: MYERS BIGEL SIBLEY & SAJOVEC, PO BOX 37428, RALEIGH, NC, 27627, US

(21) Appl. No.: 10/543,120

(22) PCT Filed: Jan. 23, 2004
(86) PCT No.: PCT/GB04/00239

§ 371 Date: Apr. 6, 2006

(30) Foreign Application Priority Data
Jan. 23, 2003 (GB) 0301560.9
Jan. 24, 2003 (GB) 0301669.8

(51) Int. Cl. A61K 31/715; A61K 31/19

(52) U.S. Cl. 514/54; 514/574

(57) ABSTRACT

There is described a pharmaceutical composition comprising a glucose polymer or a mixture of glucose polymers and optionally, salts thereof and a non-sensitising bacteriostatic agent, such as sorbic acid. There is also described a method of treatment, alleviation or prevention of HIV-1 or a related virus or other sexually transmitted diseases by the administration of a such a composition.

This invention relates to a novel pharmaceutical composition and a novel method of treatment related thereto.

In particular the invention to novel formulations of polyglucoses, such as dextrin sulphates, and to the use of such materials and compositions as agents in the topical treatment against human immunodeficiency virus type 1 (HIV-1) and related viruses and other sexually transmitted diseases (STDs).

It is known that some sulphated polysaccharides have anti-HIV activity; see, for example, European Patent Specification No. 0 240 098. This specification discloses highly sulphated oligosaccharides obtained by sulphation of dextrins of relatively low molecular weight.

It is known that dextrin sulphate has antilipaemic activity. U.S. Pat. No. 3,017,407 discloses antilipaemic agents comprising sulphated polysaccharides selected from the group consisting of corn starch dextrin and corn syrup solids containing an average of between about 5 and 15, and preferably between about 8 and 12 glucose units per molecule, containing between about 1.5 and 3, sulphate groups per molecule. There is no suggestion therein that any form of dextrin sulphate has antiviral activity.

Dextrin is a mixture of polymers of glucose and the glucose units may be substituted in one or more of the 2, 3 and 6 positions by sulphate groups. A dextrin sulphate of use in the present invention may have up to two sulphate groups per molecule.

International Patent Application No. WO 92/04904 describes the use of dextrin sulphates as anti HIV-1 agents.

Administration of dextrin sulphate to patients may reduce the viral load of HIV-1 in AIDS patients, or prevent the transmission of the HIV-1 virus and/or related viruses and other STDs in patients in general.

In the context of HIV, it is thought that three different mechanisms may operate: (i) binding of the drug to a cell surface protein on lymphocytes and monocyte derived macrophages to block viral entry, (ii) inducing the release of MIP-1α and MIP-1β from tissue macrophages, which then block viral entry into CD4+ T lymphocytes and macrophages by binding to the chemokine receptor CCR-5 (the cellular co-receptor for the virus), and (iii) an intracellular mechanism in tissue macrophages.

It has also been known for some time that dextrin sulphate gel is potentially useful as an intravaginal virucide (Stafford et al., 1997. J. Acquired Immune Deficiency Syndromes & Human Retrovirology 14: 213-218). The term “microbicide” is now preferred but is synonymous with virucide and vaginal microbicide (McCormack et al., 2001. British Medical J. 322: 410-413).

The treatment, alleviation or prevention of transmission of a sexually transmitted disease, providing the STD is not one caused by HIV, employing the topical administration of dextrin sulphate is now described in our as yet unpublished pending application GB 0130756.0.

Furthermore, it has long been desired to improve the shelf life of such polyglucose formulations. However, the microbiological status of the product must therefore take into account the site of administration, e.g. for topically administered polyglucose formulations, the physiology of the vagina, for example, the product must especially be non-sensitising to the site of administration. This is especially applicable for intravaginal application.

The healthy vagina is maintained at low pH by the symbiotic presence of lactic acid bacteria (lactobacillus sp) which metabolise secreted glycogen producing lactic acid. This maintains the vagina between pH 3.8 and pH 4.5. At this pH and with this harmless microflora, the vagina is able to exclude the invasion and infection of other harmful bacteria. Therefore, there is a need for a formulation designed to maintain the product during manufacture and distribution essentially free of gram negative bacteria, yeasts and moulds.

Thus, a preservative system would be required to be broad spectrum with activity against a wide range of bacteria and fungi and would avoid or mitigate the possibility of disrupting the healthy vaginal microflora. Such a formulation must be tailored to ensure it does not present the vagina with a product related microbiological insult capable of disrupting the healthy vaginal microflora but which at the same time does preserve the formulation per se.

We have surprisingly found a group of non-sensitising bacteriostatic agents which achieve the present invention. Sorbic acid and/or potassium sorbate are conventionally used as preservatives for the protection of foodstuffs. We have now found that this group of preservatives not only acts to preserve polyglucose compositions as hereinbefore described but also does not affect, for example, vaginal microflora.

Thus according to the invention we provide a pharmaceutical composition comprising a glucose polymer or a mixture of glucose polymers and, optionally, salts thereof, and a non-sensitising bacteriostatic agent.

By the term non-sensitising we particularly mean a bacteriostatic agent which is non-sensitising when applied topically, e.g. when applied, inter alia intravaginally, rectally or to the penis.

Preferentially, the bacteriostatic agent should be one which is effective against a variety of agents, including, but not limited to, bacteria, e.g. Gram positive bacteria and/or Gram negative bacteria; yeasts and/or moulds.

In a preferred embodiment of the invention the bacteriostatic agent is one which possesses both preservative and bacteriostatic, e.g. antimicrobial, properties. Whilst a variety of such a bacteriostatic agents may be used, a preferred such agent is sorbic acid (2,4-hexadienoic acid), or a salt thereof.

A preferred salt is an alkali metal salt, e.g. a sodium or potassium salt, or an alkaline earth metal salt, e.g. calcium. When the sorbic acid is a salt, an especially preferred salt is the potassium salt. It is within the scope of the present invention to include the use of mixtures of salts and/or a mixture of a sorbic acid salt, such as, the potassium salt and sorbic acid.

When the bacteriostatic agent is sorbic acid it may be present in a variety of isomeric forms. However, the trans-trans form of sorbic acid is most preferred.

The amount of bacteriostatic agent present in the composition of the invention may vary, depending upon, inter alia, the level of glucose polymer present, etc. Generally, the amount of bacteriostatic agent present may be from 0.01 to 1.0% w/w, preferably from 0.01 to 0.5% w/w, more preferably from 0.05 to 0.2% w/w and most preferably 0.1% w/w.

The amount of glucose polymer present may also vary, depending upon, inter alia, the nature of the polyglucose or polyglucoses. Thus the composition as hereinbefore described may comprise, for example, an aqueous composition comprising at least 1 μg/ml, preferably from 1 μg/ml to 10⁵ μg/ml, more preferably from 500 μg/ml to 10⁵ μg/ml, most preferably 1×10⁴ μg/ml (a 1% w/v solution), 2×10⁴ μg/ml (a 2% w/v solution) or 4×10⁴ μg/ml (a 4% w/v solution) of polyglucose and optionally salts thereof. The composition may especially comprise 10⁴ μg/ml or 4×10⁴ μg/ml of polyglucose and optionally salts thereof.

The composition may preferentially be packaged in a single unit dosage form. Thus the composition may be made up in, for example a sachet or ampoule comprising from 1 to 10 ml of the composition, preferably from 2 to 5 ml.

Although any conventionally known glucose polymers may be used, preferred glucose polymers or a mixture of glucose polymers, and optionally salts thereof, are those polymers described in European Patent Applications Nos. 0 115 991 and 0 153 164. Glucose polymers which may hereinafter be referred to as dextrin, glucose dextrin or dextrin polymer are intended, on all occurrences, to include optionally salts thereof, preferably the anionic salts, especially the sulphate.

Thus, the preferred glucose polymer used in the composition of the invention is dextrin sulphate. The dextrin sulphate optionally may contain at most two sulphate groups per unit. All references herein to dextrin sulphate, dextrin 2 sulphate, or D-2-S are within the scope of the aforementioned definition.

Dextrin is a mixture of polymers of glucose and the glucose units may be substituted in one or more of the 2, 3 and 6 positions by sulphate groups.

A dextrin sulphate of use in the present invention may have up to two sulphate groups per glucose unit and preferred dextrin sulphates are those having about 1, or between 0.5 and 1.5, preferably up to 1.2, for example 1.1, sulphate groups per glucose unit. More preferably, the glucose polymer is the 2- or 6-sulphate of dextrin or a mixture thereof, most preferably dextrin-2-sulphate (D-2-S) that is dextrin wherein a substantial proportion of the sulphate groups are in the 2-position, preferably greater than 75%, more preferably greater than 90%, e.g. 94%.

Moreover, the composition of the invention may include one or more buffering agents to influence the pH of the composition. A variety of conventionally known buffering agents may be used. However, preferably the buffering agent is lactic acid. The amount of buffering agent may vary and may be from 0.01 to 1.0% w/w, preferably 0.025 to 0.5% w/w, more preferably from 0.05 to 0.2% w/w, most preferably from 0.075 to 0.1% w/w and especially 0.088% w/w.

We have also found that dextrin 3-sulphates have relatively poor activity against HIV and other sexually transmitted diseases (STDs) by comparison with dextrin 2- and 6-sulphates. Hereinafter, any reference to activity against STDs shall include activity against HIV. It follows that for a given sulphate content the activity of a dextrin sulphate against STDs is inversely related to the proportion of 3-sulphation. Under most reaction conditions the 3-OH group of the glucose residue in a dextrin has been found to be less reactive than the 2-OH and 6-OH groups. Therefore, enhanced activity against STDs per sulphate group can be achieved by keeping the degree of sulphation relatively low, thereby reducing the extent of 3-sulphation.

However, in selecting a particular sulphated dextrin as an anti STD agent conflicting factors are encountered. Thus, generally speaking:

1. For a given sulphate content:
(a) the toxicity increases with increasing molecular weight, and
(b) the anti-STD activity increases with increasing molecular weight.

2. For a given molecular weight:
(a) the toxicity increases with increasing sulphate content, and
(b) the anti-STD activity increases with increasing sulphate content.

It has seemed that dextrin sulphates might in fact be unusable in practice as anti-STD agents because satisfactory anti-STD activity appeared to go hand-in-hand with unacceptable toxicity, either because the molecular weight was too high or because the sulphate content was too high.

By restricting the degree of substitution to a maximum of 2 the present invention makes it possible to produce a dextrin sulphate having adequate anti-STD activity while keeping toxicity within acceptable limits. With a relatively low degree of substitution the proportion of 3-sulphation can be kept low, so that the toxicity imported into the dextrin sulphate by 3-substitution is avoided. If a dextrin is fully substituted, i.e. to give the 2,3,6-sulphate, one-third of the sulphate groups are 3-sulphate groups, which give rise to additional toxicity out of all proportion to the extent to which they enhance the anti-STD activity. The extent to which 3-sulphation occurs when the degree of substitution is kept below 2 varies with the nature of the sulphation process, but is normally substantially less than that of 2-sulphation or 6-sulphation. An examination of the n.m.r. spectrum of a dextrin sulphate gives a sufficient indication of this for practical purposes. The total sulphate content can of course be evaluated by conventional analytical methods, normally by determining the sulphur content.

The molecular weight of dextrin sulphate of use in this invention may vary over a wide range. By way of example, dextrin sulphate of use in the present invention may have a weight average molecular weight of from 15,000 to 25,000 as determined on the dextrin used to prepare the dextrin sulphate. The technique used to determine molecular weight of the dextrin is high-pressure liquid chromatography, particularly gel permeation chromatography techniques (GPC), using chromatographic columns calibrated with dextran standards, as designated by Alsop et al, J Chromatography 246, 227-240 (1982); and/or other techniques known per se.

Dextrin sulphate can be prepared by first hydrolysing starch to produce dextrin which may then be sulphated to produce dextrin sulphate. For example, use of a trimethylamine/sulphur trioxide complex in aqueous alkaline medium gives predominantly the 2-sulphate. Treatment of dextrin with cyclamic acid in dimethylformamide gives the 6-sulphate. The 3-sulphate may be made by first acetylating dextrin, then sulphating it with trimethylamine/sulphur trioxide complex in dimethylformamide and finally removing the acetyl groups with aqueous sodium hydroxide.

It is preferred to use dextrin sulphate in which there is a low proportion of low molecular weight material. As has been mentioned above, dextrin is made by hydrolysis of starch, typically by treatment of various starches with dilute acids or with hydrolytic enzyme. Such methods produce glucose polymers with a wide range of polymerisation. The degree of polymerisation (D.P.) varies from one or two up to comparatively high numbers. The direct hydrolysis product of starch might contain up to 60% by weight of material having a D.P. less than 12. In a preferred aspect of the present invention, the dextrin derivative contains a relatively high proportion of glucose polymers of D.P. greater than 12. Preferably, the dextrin derivative contains at least 50% by weight of glucose polymers of D.P. greater than 12.

More preferably, the dextrin derivative contains less than 10% by weight of glucose polymers having a D.P. less than 12. Most preferably, the dextrin derivative contains less than 5% by weight of glucose polymers having a D.P. less than 12. Such dextrin derivatives are prepared from dextrin which has been fractionated to remove dextrin with a low D.P. Known fractionation techniques may be used, including solvent precipitation and membrane fractionation.

A method of preparing a glucose polymer mixture is described in Example 2 of GB 2154469. This mixture has a weight average molecular weight of 23,700 and contains 91.9% of polymers having a degree of polymerisation greater than 12 and 7.9% of polymers having a degree of polymerisation from 2 to 10.

It is also preferred that the dextrin derivative contains little or no material with a high molecular weight. More preferably, the dextrin derivative contains little or no material with a high molecular weight. More preferably, the dextrin derivative contains little or no material with a molecular weight greater than 40,000.

Dextrin sulphate is a particularly effective agent against HIV-1 and related viruses and other STDs. Although the mechanism of its action is not understood, it may be that dextrin sulphate acts to block the attachment of the virus to cells. It appears that because of its particular somewhat globular conformation, dextrin sulphate provides a carrier of relatively closely packed sulphate groups which can particularly effectively prevent attachment of the virus to the cell and hence entry of the virus.

Dextrin sulphate may be effective in relatively low concentrations. Furthermore, the above-mentioned globular conformation of dextrin sulphate appears to allow the material to be effective against HIV-1 and related viruses and other STDs, even with a relatively low degree of sulphation. For instance, a degree of sulphation as low as one sulphate group per glucose unit, or even lower, is found to be effective at relatively low concentrations. This has the advantage that the amount of sulphation can be kept to such a low level as to avoid the side effects and toxicity which might otherwise be experienced with highly sulphated materials.

The invention also provides an agent for use in the treatment of HIV-1 and related viruses and other STDs, the agent being dextrin sulphate which contains at most 2 sulphate groups per glucose unit and contains at least 50% of polymers of a degree of polymerisation greater than 12.

According to a further feature of the invention we provide a composition for use in the treatment, alleviation or prevention of HIV, e.g. HIV-1, or a related virus or other sexually transmitted diseases which comprises dextrin sulphates in gel form, e.g. an aqueous gel.

The composition of the invention may be administered in a variety of ways depending, inter alia, upon the nature of the disorder being treated. Thus the composition can be administered enterally (including orally), but preferably is administered parentally, for instance, intravenously, intraperitoneally or topically. However, administration topically is preferred, e.g. intravaginally, rectally or to the penis.

Most preferably the composition of the invention may be administered intravaginally. Further, the invention provides a composition comprising the above-mentioned composition, together with an inert carrier or diluent.

It is within the scope of this invention for the dextrin sulphates to be provided in powder form such that the powder may be admixed with an appropriate solvent or diluent to form a gel. Isolation of the powder may be carried out by conventional methods known per se, e.g. lyophilisation, spray drying, etc. When the isolation step includes a heating process, such as spray drying, the product may be buffered to improve its stability to thermal degradation. However, preferably the composition is presented in the form of a gel, e.g. an aqueous gel.

When the dextrin sulphate is made up in to a gel it may be administered directly to the vagina, rectum or penis. Alternatively, the dextrin sulphate gel may be administered using conventionally known prophylactic devices. A preferred prophylactic device is a conventionally known condom, e.g. a condom which may be coated, either internally or externally with a dextrin sulphate gel.

The bacteriostatic agent used in the composition of the invention may be effective at a variety of pHs. However, preferably, the bacteriostatic agent is one which is effective at a low pH, for example, vaginal pH, that is, as hereinbefore described, a pH of 3.8 to 4.5. Thus, the use of a bacteriostatic agent effective at such a pH is especially advantageous for use in an intravaginally administered gel.

Thus according to a further aspect of the invention we provide a method of treatment or alleviation of HIV-1 or a related virus or a sexually transmitted disease which comprises the administration of a composition as hereinbefore described.

Thus we especially provide a method which comprises the topical administration of a composition of the invention. Most preferably the topical administration will include administration in, around or on the genitalia, the genito urinary tract and/or the rectum. Thus, the method of the invention may comprise intravaginal administration of dextrin sulphate, penile administration or rectal administration of a composition of the invention.

We especially provide a method which comprises the administration of a composition of the invention intravaginally. The method of the invention is particularly advantageous in the treatment, alleviation or prevention of sexually transmitted HIV-1 and/or related viruses.

The method of the invention is particularly advantageous in the treatment, alleviation or prevention of sexually transmitted diseases.

However, the method of the invention may comprise the treatment of any known STD or combination of STDs. Thus, the STD may comprise any conventionally known STD, such as a viral disease, a bacterial disease or a protozoal disease. However, specific STDs which may be mentioned are bacterial vaginosis, chlamydia, genital herpes, genital warts, gonorrhoea, syphilis and trichomoniasis and Candida. When the invention comprises the treatment, alleviation or prevention of HPV, this may be manifested as the treatment, alleviation or prevention of genital warts.

The dosage of dextrin sulphate used in the method of the invention may vary, depending upon, inter alia, the nature and severity of the disorder. However we have found that a suitable dosage comprises administering from 1 to 10 ml of a formulation comprising at least 1 μg/ml, preferably 1 μg/ml to 10⁵ μg/ml, more preferably from 500 μg/ml to 10⁵ μg/ml, most preferably 1×10⁴ μg/ml (a 1% w/v solution), 2×10⁴ μg/ml (a 2% w/v solution) or 4×10⁴ μg/ml (a 4% w/v solution). Preferably, the method of the invention comprises administration of from 2 to 5 ml of a formulation as hereinbefore described. The formulation may be administered at any time, however, for the formulation to be most efficacious it is preferred that the formulation be administered immediately before or shortly before sexual activity.

We further provide the use of dextrin sulphates in the manufacture of a composition for the treatment, alleviation or prevention of HIV-1 or a related virus or other sexually transmitted disease.

The method of the invention is advantageous in that, inter alia, the dextrin sulphate has little or no spermicidal activity, whilst still possessing the desired microbicidal activity.

The following examples illustrate methods for the laboratory preparation of dextrin sulphate:

### EXAMPLE 1

### Preparation of Dextrin 3-sulphate

16.2 of the aforementioned dextrin of Example 2 of GB 2,154,469 in dimethylformamide (150 ml) was stirred and heated until dissolved, then cooled to ambient temperature. Acetic anhydride (23 ml, 0.24 mole) was added slowly with stirring. A transient precipitation occurred when this had redissolved, triethylamine (25 ml, 0.18 mole) was added and the mixture stirred for 2 days. The solution was then poured in a thin stream with stirring into water (700 ml), the precipitate was filtered off, washed with water and dried to give 23 g of white powder. The acetylated dextrin (12.3 g) in dimethylformamide (75 ml) was stirred until dissolved then trimethylamine sulphur trioxide complex (15 g) was added and the mixture was stirred at ambient temperature overnight. Further trimethylamine sulphur trioxide complex (15 g) was added and the mixture was stirred at ambient temperature overnight. Further trimethylamine sulphur trioxide (10 g) was added and the mixture heated at 60 C for 3 hours. The solution was cooled. and poured into acetone (500 ml) to give a sticky residue. The supernatant was decanted and the residue kneaded with fresh acetone (50 ml) and then the supernatant decanted. The residue was dissolved in water (150 ml) and the remaining acetone stripped off under vacuum. A solution of NaOH (5 g) in water (10 ml) was added giving trimethylamine gas. The strongly basic solution was stored for 2 h, dialysed against water for 4 days and freeze dried, to give 10.2 g. The I.R. spectrum showed peaks for acetate (1750 ml) and NaOH (1 g) in water added and the mixture stirred 3 h at ambient temperature. The solution was poured into ethanol (300 ml), the supernatant was decanted and the sticky residue kneaded with fresh ethanol (150 ml) to give a solid. The solid was filtered off, washed with methanol and dried to a brown powder. The powder was dissolved in water (200 ml) and decolourising charcoal (5 g) added. The solution was warmed then filtered twice and freeze-dried to give 7.2 g, sulphate, 46.9%.

### EXAMPLE 2

### Preparation of Dextrin 6-sulphate

10 g of the same dextrin as in Example 1 in dimethylformamide (100 ml) was heated and stirred at 78° C. When the dextrin had all dissolved cyclamic acid (22.5 g) was added and the reaction continued for 1.5 h. A solution of NaOH (5 g in water (5 ml) and ethanol (50 ml) was added and the mixture poured into diethyl ether (400 ml). The solid was filtered off, washed with ether and air dried. The solid was dissolved in water (100 ml), sodium acetate (50 g) added and the solution dialysed against water for 4 days then freeze dried to give 15.4 g, sulphate 47.2%.

### EXAMPLE 3

### Preparation of Dextrin 2-sulphate

40 g of the same dextrin as in Example 1 in distilled water (150 ml) were stirred in a round bottomed flask at 30° C. When the dextrin had all dissolved trimethylamine sulphur trioxide (42 g) were added to the solution. The reaction mix was stirred for thirty minutes. Sodium hydroxide (62.5 ml @40% w/v) was added dropwise to the reaction mix over a period of one hour. The reaction mix was then stirred for a further hour. Activated carbon was added to the flask and the solution was stirred for a further hour and then filtered under vacuum. The resultant solution was dialysed for one day against tap water and one day against distilled water. The dialysed solution was then concentrated by evaporation at reduced pressure. The concentrated solution containing 40 g of dissolved solids was freeze dried to give a product containing 40% w/w (wrt. dry solids) sulphate.

The products of Examples 1, 2 and 3 have been identified as the 3-, 6- and 2-sulphates respectively by examination of their n.m.r. spectra.

The 13C n.m.r. spectrum of the original dextrin shows six lines. These can mostly be assigned, by reference to standard compounds, as: 100.3, 77.6, C-4; 73.9, C-3; 72.2 and 71.8, C2 and C-5; 61.1, C-6.

The n.m.r. spectra of both the 3- and 6-sulphates of glucose have been reported (S. Honda, Y. Yuki and K. Tahiura, Carbohydrate Research (1973) Volume 28, pages 130 to 150) and compared to the free sugars. Thus, 3-O-sulphation was observed to cause 8.5 or 9.5 ppm downfield shift for C-3, a 1.1 ppm upfield shift for C-2 and 2.2 ppm upfield shift for C-4, but little change for other positions. For 6-O-sulphation, a downfield shift of 6.2 ppm was observed for C-6 and upfield shifts of 1.7 ppm for C-5 and 0.3 ppm for C-4, with little change in other positions.

The n.m.r. spectrum of the product of Example 1 shows a strong signal at 61.1 ppm, characteristic of unmodified C-6-OH. Prominent new signals have appeared at 82.2 and 82.5 ppm. These are close to the chemical shift of 82.7 ppm reported for C-3 in D-glucose-3-sulphate and are therefore assigned to dextrin-3-sulphate. This assignment is supported by the virtual disappearance of the signal at 77.6 ppm in the original dextrin for C-4. Substitution at 0-3 is expected to cause an upfield shift of the signal for C-4, taking it under the envelope of other signals. New peaks at 70.2 and 70.8 ppm are attributed to C-2 in a 3-sulphate by upfield shift from the original position at 72.2 or 71.8 ppm. The C-1 region shows six closely spaced lines between 100.1 and 98.3 ppm slightly upfield from that in the original dextrin. From this data it appears that the product of Example 1 is sulphated almost entirely in the 3-position. The n.m.r. spectrum of the product of Example 2 shows that the original C-6 peak at 61.1 ppm has greatly diminished and new peaks have appeared at 67.5 ppm and 69.3 ppm, for C-6-O-sulphate (6.4 ppm downfield shift) and for C-5 adjacent to 6-O sulphate (2.5 or 2.9 upfield shift) respectively. This data indicates that the product of Example 2 is substituted primarily in the 6-position.

The n.m.r. spectrum of the product of Example 3, in comparison with that of the orginal dextrin, shows a major signal for unsubstituted C-6-OH at 61.1 ppm, unperturbed C-4 signal at 78.1 ppm, indicating free 3-OH and the major C-1 signal moved upfield to 99.8 ppm from its original position at 100.3 ppm. From this data it appears that the product of Example 3 is substituted primarily in the 2-position.

In the following formulation examples the dextrin sulphate used is dextrin-2-sulphate, which is dextrin substituted primarily in the 2-position.

### EXAMPLE 4

1% w/w Dextrin Sulphate/Sorbic acid gel formulation

| Dextrin Sulphate | 10.0 | g (dry weight) |
|---|---|---|
| Carbopol | 40.0 | g (dry weight) |
| Lactic acid | 0.88 | g |
| Sorbic acid | 0.5 | g |
| Sodium hydroxide (BP), 40% w/w Solution: | for pH adjustment | |
| Sterile water (Water for Injection (W.F.I)): | 948.6 | g |

Preservative efficacy data for example 4 is given in Table I below.

TABLE I
Mean Count per ml sample after

| | 0 hr | 7 days | 14 days | 21 days | 28 days |
|---|---|---|---|---|---|
| C. albicans | 1.1 × 10⁶ | 2.8 × 10² | <5 | <5 | <5 |
| A. niger | 6.2 × 10⁵ | 4.5 × 10¹ | <5 | <5 | <5 |
| P. aeruginosa | 1.8 × 10⁶ | <5 | <5 | <5 | <5 |
| S. aureus | 1.3 × 10⁶ | <5 | <5 | <5 | <5 |
| E. coli | 8.9 × 10⁵ | <5 | <5 | <5 | <5 |

### EXAMPLE 5

1% w/w Dextrin Sulphate/Sorbic acid gel formulation

| Dextrin Sulphate | 10.0 | g (dry weight) |
|---|---|---|
| Carbopol | 40.0 | g (dry weight) |
| Lactic acid | 0.88 | g |
| Sorbic acid | 1.0 | g |
| Sodium hydroxide (BP), 40% w/w Solution: | for pH adjustment | |
| Sterile water (Water for Injection (W.F.I)): | 948.1 | g |

Preservative efficacy data for example 5 is given in Table II below.

TABLE II
Mean Count per ml

| | 0 hr | 7 days | 14 days | 21 days | 28 days |
|---|---|---|---|---|---|
| C. albicans | 1.1 × 10⁶ | <5 | <5 | <5 | <5 |
| A. niger | 7.0 × 10⁵ | <5 | <5 | <5 | <5 |
| P. aeruginosa | 1.8 × 10⁵ | <5 | <5 | <5 | <5 |
| S. aureus | 1.3 × 10⁶ | <5 | <5 | <5 | <5 |
| E. coli | 9.1 × 10⁵ | <5 | <5 | <5 | <5 |

### EXAMPLE 6

4% w/w Dextrin Sulphate/Sorbic acid gel formulation

| Dextrin Sulphate | 40.0 | g (dry weight) |
|---|---|---|
| Carbopol | 40.0 | g (dry weight) |
| Lactic acid | 0.88 | g |
| Sorbic acid | 0.5 | g |
| Sodium hydroxide (BP), 40% w/w Solution: | for pH adjustment | |
| Sterile water (Water for Injection (W.F.I)): | 918.6 | g |

Preservative efficacy data for example 6 is given in table III below.

TABLE III
Mean Count per ml

| | 0 hr | 7 days | 14 days | 21 days | 28 days |
|---|---|---|---|---|---|
| C. albicans | 1.1 × 10⁶ | 6.2 × 10² | <5 | <5 | <5 |
| A. niger | 6.4 × 10⁵ | <5 | <5 | <5 | <5 |
| P. aeruginosa | 1.9 × 10⁵ | <5 | <5 | <5 | <5 |
| S. aureus | 1.3 × 10⁶ | <5 | <5 | <5 | <5 |
| E. coli | 9.0 × 10⁵ | <5 | <5 | <5 | <5 |

### EXAMPLE 7

### 4% w/w Dextrin Sulphate/Sorbic Acid Gel Formulation

Carbopol 974-P: 40.0 g (dry weight)

Dextrin Sulphate: 40.0 g (dry weight)

Lactic Acid (BP): 0.88 g

Sorbic Acid (Ph Eur): 1.0 g

Sodium hydroxide (BP), 40% w/w Solution: for pH adjustment

Sterile water (Water for Injection (W.F.I)): 918.1 g

Preservative efficacy data for example 7 is given in Table IV below.

TABLE IV
Mean Count per ml

| | 0 hr | 7 days | 14 days | 21 days | 28 days |
|---|---|---|---|---|---|
| C. albicans | 1.1 × 10⁶ | <5 | <5 | <5 | <5 |
| A. niger | 6.7 × 10⁵ | <5 | <5 | <5 | <5 |
| P. aeruginosa | 1.8 × 10⁶ | <5 | <5 | <5 | <5 |
| S. aureus | 1.3 × 10⁶ | <5 | <5 | <5 | <5 |
| E. coli | 8.5 × 10⁵ | <5 | <5 | <5 | <5 |

1. A pharmaceutical composition comprising a polyglucose or a mixture of polyglucose molecules, and optionally, salts thereof and sorbic acid, or a salt thereof.

2. The pharmaceutical composition according to claim 1, wherein the composition is non-sensitizing when applied topically intravaginally, rectally, to the penis, to areas around or on the genitalia, to the genito urinary tract and/or to the rectum.

3. The pharmaceutical composition according to claim 1, wherein the composition is effective against Gram positive bacteria, Gram negative bacteria, yeasts and/or molds.

4. The pharmaceutical composition according to claim 1, wherein the sorbic acid salt is an alkali metal or alkali earth metal salt.

5. The pharmaceutical composition according to claim 4, wherein the salt is a potassium or calcium salt.

6. The pharmaceutical composition according to claim 1, wherein the sorbic acid, or a salt thereof, is the trans-trans form.

7. The pharmaceutical composition according to claim 1, wherein the sorbic acid, or a salt thereof is present in an amount of from 0.01 to 1.0% w/w.

8. The pharmaceutical composition according to claim 1, wherein the composition is buffered to vaginal pH.

9. The pharmaceutical composition according to claim 8, wherein the composition is buffered to a pH of from 3.8 to 4.5.

10. The pharmaceutical composition according to claim 1 further comprising a buffering agent.

11. The pharmaceutical composition according to claim 10, wherein the buffering agent is lactic acid.

12. The pharmaceutical composition according to claim 10, wherein the buffering agent is present in an amount of from 0.01 to 1.0% w/w.

13. The pharmaceutical composition according to claim 1, wherein the composition is formulated in an aqueous gel, gel or powder form.

14. The pharmaceutical composition according to claim 1, wherein the polyglucose, or a salt thereof, is present in an amount of at least 1 μg/ml and up to 10⁵ μg/ml.

15. The pharmaceutical composition according to claim 1, wherein the composition comprises a unit dosage form comprising from 1 to 10 ml of the composition.

16. The pharmaceutical composition according to claim 1, wherein the polyglucose polymer is a salt.

17. The pharmaceutical composition according to claim 16, wherein the salt is an anionic salt.

18. The pharmaceutical composition according to claim 1, wherein the polyglucoses are dextrins, or salts thereof.

19. The pharmaceutical composition according to claim 18, wherein the salt is a sulphate.

20. The pharmaceutical composition according to claim 19, wherein the polyglucose is a dextrin sulphate.

21. The pharmaceutical composition according to claim 20, wherein the dextrin sulphate comprises at most two sulphate groups per unit.

22. The pharmaceutical composition according to claim 19, wherein the glucose units of the dextrin are substituted in one or more of the 2, 3 and 6 positions by sulphate groups.

23. The pharmaceutical composition according to claim 22, wherein a substantial proportion of the sulphate groups are in the 2-position.

24. The pharmaceutical composition according to claim 23, wherein greater than 70% of the sulphate groups are in the 2-position.

25. The pharmaceutical composition according to claim 1, wherein up to 60% by weight of the polyglucose has a D.P. less than 12.

26. The pharmaceutical composition according to claim 1, wherein the polyglucose contains at least 50% by weight of glucose polymers of D.P. greater than 12.

27. The pharmaceutical composition according to claim 26, wherein the polyglucose comprises less than 10% by weight of glucose polymers having a D.P. less than 12.

28. The pharmaceutical composition according to claim 1, wherein the polyglucose contains little or no material with a high molecular weight.

29. The pharmaceutical composition according to claim 28, wherein the polyglucose contains little or no material with a molecular weight greater than 40,000.

30. The pharmaceutical composition according to claim 13, wherein the composition is a gel and is administered in a prophylactic device.

31. The pharmaceutical composition according to claim 30, wherein the prophylactic device is a condom.

32. The pharmaceutical composition according to claim 1, wherein the composition comprises an inert carrier or diluent.

33. The pharmaceutical composition according to claim 1, wherein the composition is an agent for use in the treatment of HIV-1 and related viruses or other sexually transmitted diseases.

34. The pharmaceutical composition according to claim 1, wherein the composition is formulated for enteral or parenteral administration.

35. A method of treating, alleviating or preventing HIV-1 or a related virus or other sexually transmitted diseases, comprising administering a composition of claim 1.

36. (canceled)

37. The method according to claim 35, wherein the composition is suitable for topical administration.

38. The method according to claim 37, wherein the topical administration comprises administration in, around or on the genitalia, the genito urinary tract and/or the rectum.

39. The method according to claim 38, wherein the composition is suitable for intravaginal administration, penile administration or rectal administration.

40. The method according to claim 35, wherein the composition is suitable for the treatment of an STD or combination of STDs.

41. The method according to claim 40, wherein the STD comprises bacterial vaginosis, chlamydia, genital herpes, genital warts, gonorrhea, syphilis, trichomoniasis, Candida and combinations thereof.

42. The method according to claim 35, wherein the method further comprises administering from 1 to 10 ml of the composition.

43. The method according to claim 35, wherein the polyglucose, or a salt thereof, is present in an amount of at least 1 μg/ml and up to 10⁵ μg/ml.

44. The method according to claim 35, wherein the method comprises administering the composition prior to sexual activity.
